(19) United States
(12) Patent Application Publication
(10) Pub. No.: US 2026/0118265 A1
(43) Pub. Date: Apr. 30, 2026

(54) SYSTEM AND METHOD FOR THE ANALYSIS OF NATURAL GAS

(71) Applicant: Coolaimate, Inc., Altadena, CA (US)

(72) Inventor: Yi ZHANG, Pasadena, CA (US)

(21) Appl. No.: 19/367,730

(22) Filed: Oct. 23, 2025

Related U.S. Application Data
(60) Provisional application No. 63/711,283, filed on Oct. 24, 2024.

(51) Int. Cl. G01N 21/39; G01N 33/00

(52) U.S. Cl. CPC G01N 21/39; G01N 33/0047; G01N 2201/06113

(57) ABSTRACT

A system and method for the analysis of natural gas. In some embodiments, the system, includes: a natural gas analyzer, including: a coherent light source; a sample chamber configured to contain a sample of natural gas; and a photodetector, the photodetector being configured to measure a transmission fraction, through the sample chamber, of light from the coherent light source; the coherent light source being configured to selectively emit light at a first wavelength, a second wavelength, and a third wavelength; the first wavelength and the second wavelength being within the 1/e² width of a methane absorption line; the third wavelength differing from the first wavelength by at least 5 nm; and the natural gas analyzer being configured to measure the transmission fraction of the sample chamber at the first wavelength, the second wavelength, and the third wavelength.

### CROSS-REFERENCE TO RELATED APPLICATIONS

The present application claims priority to and the benefit of U.S. Provisional Application No. 63/711,283, filed October 24, 2024, entitled "SYSTEM AND METHOD FOR ANALYZING PROPERTIES OF NATURAL GAS", the entire content of which is incorporated herein by reference.

### FIELD

One or more aspects of embodiments according to the present disclosure relate to natural gas, and more particularly to a system and method for the analysis of natural gas.

### BACKGROUND

Natural gas is a key fuel gas with a wide range of uses, from cooking, space heating, and transportation to electricity generation. It is a cleaner alternative to traditional fossil fuels like coal and oil, producing less carbon dioxide and pollutants when burned. Natural gas comprises mainly methane, ethane, and carbon dioxide, as well as heavier hydrocarbons at lower levels. As a measure of quality control, the energy content of natural gas, expressed in British thermal units (BTUs) or calorific values (CVs), is characterized throughout the distribution chain. The diversification of sources of natural gas such as biomethane and imports from liquid natural gas (LNG) tankers further boosts the need for BTU/CV measurements. The BTU/CV of natural gas is calculated based on its composition and physical constants of constituent components.

It is with respect to this general technical environment that aspects of the present disclosure are related.

### SUMMARY

According to an embodiment of the present disclosure, there is provided a system, including: a natural gas analyzer, including: a coherent light source; a sample chamber configured to contain a sample of natural gas; and a photodetector, the photodetector being configured to measure a transmission fraction, through the sample chamber, of light from the coherent light source; the coherent light source being configured to selectively emit light at a first wavelength, a second wavelength, and a third wavelength; the first wavelength and the second wavelength being within the 1/e² width of a methane absorption line, i.e. the wavelength range between two points on the methane absorption line where the absorption is 1/e² (approximately 13.5%) of the peak absorption; the third wavelength differing from the first wavelength by at least 5 nm; and the natural gas analyzer being configured to measure the transmission fraction of the sample chamber at the first wavelength, the second wavelength, and the third wavelength.

In some embodiments, the coherent light source includes: a first laser, configured to emit light at the first wavelength and at the second wavelength; a second laser, configured to emit light at the third wavelength; and a wavelength multiplexer for combining light emitted by the first laser with light emitted by the second laser.

In some embodiments, the system further includes a photonic integrated circuit, including the first laser, the second laser, and the wavelength multiplexer.

In some embodiments, the system further includes a processing circuit.

In some embodiments, the processing circuit is configured to turn on or off the first laser and the second laser.

In some embodiments, the processing circuit is configured to control the wavelength of the first laser.

In some embodiments, the system includes eight lasers including the first laser and the second laser.

In some embodiments, the system includes 15 lasers including the first laser and the second laser.

In some embodiments, the first wavelength is between 1620 nm and 1850 nm, and the third wavelength is between 1620 nm and 1850 nm.

In some embodiments, the first wavelength is between 1650 nm and 1720 nm, and the third wavelength is between 1650 nm and 1720 nm.

In some embodiments, the system is configured to calibrate each of the first wavelength, the second wavelength, and the third wavelength against a methane absorption line.

In some embodiments, the system is configured to estimate the concentration of alkanes with molecular formula CₙH₂ₙ₊₂, with n ranging from 1 to 5 including isomers.

In some embodiments, the coherent light source includes a continuously tunable laser, the continuously tunable laser being capable of emitting light at each of the first wavelength, the second wavelength, and the third wavelength.

In some embodiments, the continuously tunable laser includes: a semiconductor gain medium; and a first mirror, the first mirror being a tunable mirror.

In some embodiments, the first mirror includes a first resonant element and a second resonant element in a vernier configuration.

In some embodiments, the system includes a photonic integrated circuit, including: the semiconductor gain medium; the first mirror; a phase tuning section; and a second mirror.

In some embodiments, the system further includes a temperature control circuit for controlling the temperature of the photonic integrated circuit.

In some embodiments, the system further includes a processing circuit.

In some embodiments, the processing circuit is configured to control the wavelength of the continuously tunable laser.

### BRIEF DESCRIPTION OF THE DRAWINGS

These and other features and advantages of the present disclosure will be appreciated and understood with reference to the specification, claims, and appended drawings wherein:

FIG. 1 is a graph of absorption cross-sections as a function of wavelength, according to an embodiment of the present disclosure;

FIG. 2 is a block diagram of a natural gas analyzer, according to an embodiment of the present disclosure;

FIG. 3A is a schematic drawing of a portion of a light source, according to an embodiment of the present disclosure;

FIG. 3B is a schematic drawing of a portion of a light source, according to an embodiment of the present disclosure; and

FIG. 4 is a flow chart of a method for analyzing the composition of natural gas, according to an embodiment of the present disclosure.

### DETAILED DESCRIPTION

The detailed description set forth below in connection with the appended drawings is intended as a description of exemplary embodiments of a system and method for the analysis of natural gas provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

Compositional analysis of natural gas may be performed by gas chromatography, as detailed in Gas Producers Association (GPA) 2261, titled "Analysis of Natural Gas and Similar Gaseous Mixtures by Gas Chromatography." Physical properties of hydrocarbons are tabulated in various sources, such as the GPA-2145-16, "Table of Physical Properties of Hydrocarbons and Other Compounds."

The chromatography column is central to a natural gas chromatograph (NGC). It consists of a metal tube packed with column material—small particulates coated with a thin liquid solvent known as the stationary phase. Hydrocarbon molecules in a natural gas sample are carried by a carrier gas and travel through the column at different speeds depending on their volatility. Hence, they are separated in elution time. A natural gas chromatograph may have a multi-column design to achieve an analysis time of about 5 minutes, significantly reduced compared to the 20 to 30 minutes required by a single column. For example, the first column separates hexane and heavier hydrocarbons and backflushes them as one composite called C6+; the second column separates medium-weight molecules, propane, pentane, and butane; the third column separates the lightest molecules remaining in the sample, i.e., methane, ethane, nitrogen, and carbon dioxide. A thermal conductivity detector (TCD) quantifies the concentration of the molecules after they exit the column. The TCD uses two thermistors in a Wheatstone bridge configuration. The carrier gas flows through the reference thermistor, while the sample and carrier gas mixture flows through the analyte thermistor. A small temperature difference develops due to the difference in thermal conductivity between the carrier and the analyte gas, which is detected and amplified by electronics. Helium is a commonly used carrier gas because of its large thermal conductivity. A 50-liter bottle lasts 6 months to a year depending on the NGC model. The column and detector degrade over time. NGCs are typically checked against a reference gas daily to ensure they work properly at custody transfer sites. Consumables and accessories account for over 50% of the overall gas chromatography market. NGC manufacturers partially mitigate the challenge by designing the units for easy service access or module replacement. Hence a natural gas analyzer, based on optical technologies, that requires little or no consumables and little maintenance, and has a shorter response time will be appreciated, as described in GPA 2119, ‘Compositional Analysis by Optical Spectroscopy’.

Near-infrared (NIR) spectrometers may be used for process control and chemical analysis. An NIR spectrometer may use a broadband optical source, such as a tungsten halogen lamp, and a dispersive element such as a grating to resolve the spectral response of the material under test. The wavelength resolution of NIR spectrometers ranges from 2 nm to 20 nm, which is useful for analyzing solids and liquids but insufficient to resolve rovibrational transitions of gas molecules.

Gas analyzers based on tunable diode laser absorption spectroscopy (TDLAS) may be used for detecting trace-level moisture and hydrogen sulfide in natural gas. A TDLAS analyzer uses a telecom-grade distributed feedback (DFB) near-infrared laser and photodetector. A multi-pass cell extends the optical path length for stronger absorption, which varies from less than a meter to multiple kilometers depending on the mirror reflectivity. The linewidth of a DFB is about 10 MHz, much smaller than the width of a gas molecule rovibrational absorption line, which is on the order of 3 GHz at atmospheric pressure. Thus, when the DFB is tuned by changing its injection current, the profile of a gas absorption line can be fully resolved. Gas concentration can then be calculated from the absorbance according to Beer's law, using either the peak absorbance or the 'area under the curve'. SpectraSensors Inc., a spinoff of NASA Jet Propulsion Lab (JPL) and now part of Endress+Hauser, pioneered making TDLAS moisture analyzers for natural gas in the early 2000s. Technology developments discussed in US Pat. No. 6,657,198; 8,547,554; 10,739,255; and research such as that published in an article titled "New process gas analyzer for the measurement of water vapor concentration" by A. Amerov, M. Maskas, W. Meyer, R. Fiore, and K. Tran at 52nd Instrumentation, System, and Automation Society (ISA) Analysis Division Symposium in 2007, led to multiple TDLAS gas analyzers on the market.

TDLAS analyzers require no consumables and low maintenance. The typical response time is a few seconds. However, a DFB can be tuned typically over just 1 to 2 nm in wavelength by current. Because propane and heavier hydrocarbons have broad absorption spectra beyond the tuning range of a DFB, conventional TDLAS analyzers may not be capable of analyzing the composition of natural gas. Each of the components of interest may be an alkane. Alkanes may be classified into sets of one or more isomers, each such set having a respective chemical formula given by CₙH₂ₙ₊₂.

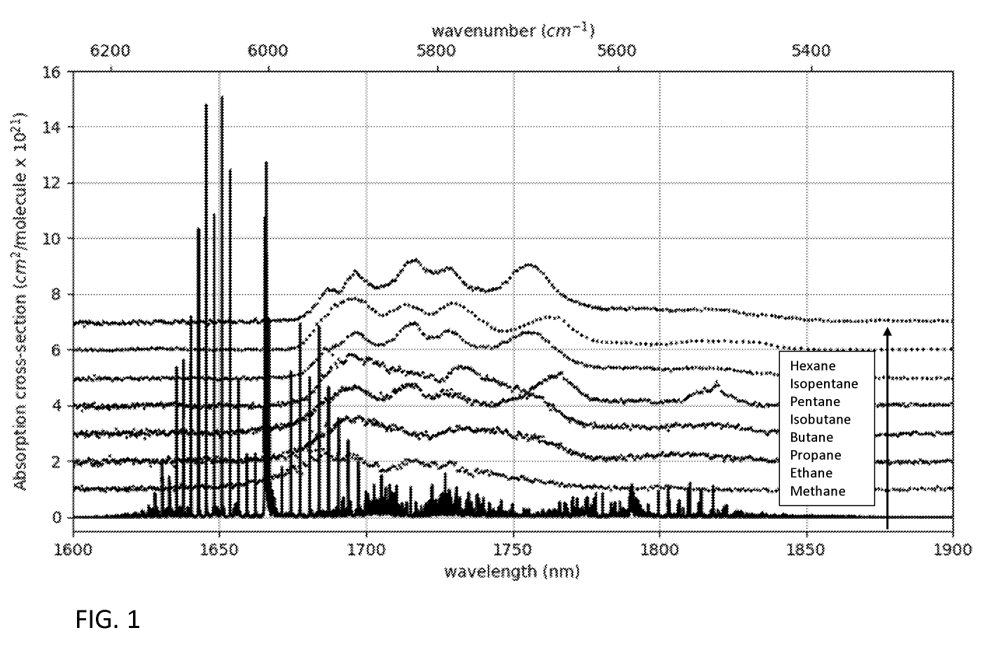

FIG. 1 shows the absorption cross-section of methane, ethane, propane, butane, isobutane, pentane, isopentane, and hexane, as a function of wavelength. In the graph of FIG. 1, the vertical axis label shows the absorption cross-section of methane; the absorption cross-section of each other species is offset vertically (to reduce the extent to which the graphed curves obscure each other) by an integer multiple of 1 cm² per 10²¹ molecules, with the integer being 1 for ethane, 2 for propane, 3 for butane, 4 for isobutane, 5 for pentane, 6 for isopentane, and 7 for hexane. Hydrocarbon molecules exhibit rovibrational transitions in the NIR region between 1600 nm and 1850 nm, as illustrated in FIG. 1. A photonic integrated circuit (PIC) may be constructed to emit light at multiple wavelengths, one at a time (e.g., sequentially) in this range (e.g., at wavelengths spaced apart by more than 2 nm), thus addressing the narrow wavelength range of a DFB and enabling a system and method to analyze the composition and other properties of natural gas. Such an analyzer may use consumables at a low rate and may require little maintenance. A measurement may be conducted every few seconds. As used herein, a photonic integrated circuit is an optical system comprising a substrate (e.g., a substrate composed of silicon, indium phosphide, or silica) which includes (e.g., fabricated on, or bonded to) its surface, at least one optical waveguide. A photonic integrated circuit may also include passive structures (e.g., filters, or wavelength multiplexers) made from optical waveguides or active elements (e.g., a gain section 320 (discussed in further detail below)) which may be bonded to the surface of the photonic integrated circuit in such a manner that a waveguide of the active element is aligned with a waveguide of the photonic integrated circuit, so that light may couple between the waveguides.

Methane has sharp absorption lines at its P, Q, and R branches between 1620 nm and 1700 nm, and nearly continuum absorption cross-sections between 1700 nm and 1850 nm, with amplitude comparable to those of heavier hydrocarbons. Since the concentration of methane is typically above 90% in pipeline natural gas, it will dominate the absorption spectrum of natural gas. The concentration of methane can be determined by TDLAS techniques using one of its sharp absorption lines. Broad spectra of ethane and heavier hydrocarbons can be sampled at multiple wavelengths and corresponding concentrations can be determined by a multivariate analysis such as partial least square regression (PLSR).

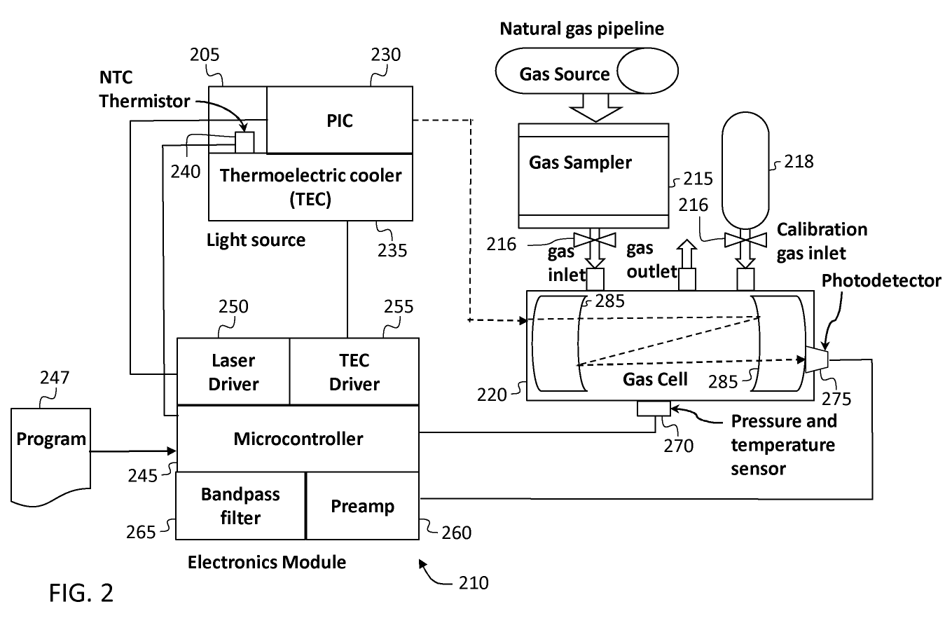

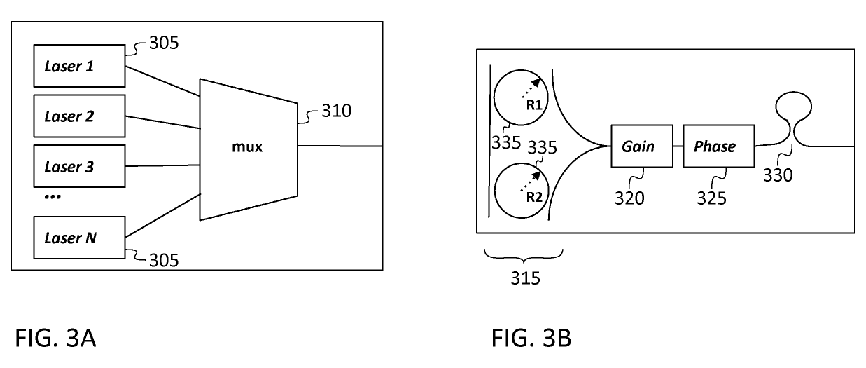

FIG. 2 shows a block diagram of a natural gas analyzer in some embodiments. The natural gas analyzer includes a light source 205, a control circuit or “electronics control module” 210, a gas sampler 215, and a gas cell, or “sample chamber” 220. In FIG. 3, electrical connections are represented by solid black lines, optical beams are represented by dashed lines, and gas tubes are represented by filled arrows. The light source 205 contains a photonic integrated circuit (PIC) 230, a thermoelectric cooler (TEC) 235, and a temperature sensor 240 (e.g., negative thermal coefficient (NTC) thermistor). The control circuit 210 contains a microcontroller unit (MCU) 245, a laser driver 250 (for supplying a drive current to a laser of the photonic integrated circuit 230), a TEC driver 255, a preamplifier 260, a and a bandpass filter 265. The microcontroller unit 245 may execute a program 247 stored in its firmware. The MCU may contain a digital core, analog-to-digital converters (ADCs), digital-to-analog converters (DACs), and other circuits. It may interface with the laser driver 250, the TEC driver 255, the temperature sensor 240, the preamplifier 260, and a pressure and temperature sensor 270 of the gas cell 220. The preamplifier 260 may amplify a signal produced by a photodetector 275 of the gas cell 220 (as discussed in further detail below).

The temperature of the photonic integrated circuit 230 may be actively controlled using the temperature sensor 240 and the thermoelectric cooler 235. For example, the microcontroller unit 245 may measure the resistance of the temperature sensor 240 and adjust the output of the TEC driver 255 according to a feedback algorithm (e.g., a proportional integral and derivative (PID) algorithm) in its program to maintain the temperature of the photonic integrated circuit 230 at a suitable set point. The microcontroller unit 245 may also control the laser driver to set the laser drive current, which may determine the laser output power and wavelength. The microcontroller unit 245 may also monitor the pressure and temperature of the gas cell, which may be used to estimate the pressure broadening of gas absorption lines and determine temperature dependent absorption coefficients.

The gas sampler 215 may include (e.g., consist of) a pressure regulator and mass flow controller, which samples a small portion of the gas in a gas pipeline 280 and injects it into the gas cell 220. The light emitted by the photonic integrated circuit 230 is guided into the gas cell 220, interacts with the gas through a single pass or multiple passes, and is then received by a photodetector 275 of the gas cell 220. The light intensity is attenuated, after passing through the gas cell, according to the absorbing properties of the gas. The gas cell may include one or more mirrors (285) to extend the effective optical path length beyond the physical dimensions of the cell. For example, multiple-pass absorption cells such as Herriot or integrated cavity output spectroscopy (ICOS) cells could be used to achieve effective absorption path lengths from meters to kilometers.

In operation, the light source 205 may generate a number of different wavelengths one at a time, and the natural gas analyzer may infer, from the photocurrent produced by the photodetector 275 when illuminated by the light after it is partially transmitted through the gas in the gas cell 220 (and attenuated, e.g., partially absorbed , by the gas in the gas cell 220), the transmission fraction, through the gas, of light at each wavelength. The set of transmission fractions, at the set of wavelengths for which the transmission fraction was measured, may be referred to as the spectral transmission fraction. The natural gas analyzer may then infer the composition of the natural gas from the spectral transmission fraction. This may be accomplished, for example, using the absorption cross sections of the components (e.g., as illustrated in FIG. 1) and a suitable fitting method, e.g., partial least square regression (PLSR) method.

FIGS. 3A and 3B are schematic drawings of portions of the light source 205 that may be constructed on the photonic integrated circuit 230. In the embodiment of FIG. 3A, a plurality of lasers 305, each having a different nominal wavelength, produce light that is then combined using a multiplexer 310 (which may be an arrayed waveguide grating (AWG) or an echelle grating, or, for relatively few (e.g., 16, or 8, or fewer) wavelengths, a cascaded Mach-Zehnder interferometer multiplexer). Each laser 205 may be a DFB laser; the lasers 205 may have different nominal wavelengths as a result of having different grating pitches. The microcontroller unit 245 may control each of the lasers 305, turning on one laser 305 at a time, and, if desired, adjusting the wavelength of the laser 305 that is turned on by adjusting its drive current. In some embodiments the wavelength of each laser may also be tunable by adjusting the temperature of the gratings of the laser. The number of lasers may be at least as great as the number of parameters to be estimated; for example, if the ratio of (i) the molecular density (the number of molecules per unit volume, which may also be referred as the concentration) of each non-methane component, of the eight components shown in FIG. 1 to (ii) the molecular density of methane is to be estimated, the light source 205 may include at least seven lasers. Some embodiments include a larger number of lasers (e.g., between 8 and 200 lasers), which may enable the natural gas analyzer to produce more accurate estimates of the composition of the gas.

In the embodiment of FIG. 3B, the light source 205 includes a single widely tunable laser. The laser includes a back mirror 315 (which may be a tunable mirror, as shown), a gain section 320, a phase tuning section 325, and an output mirror 330, which may be a loop mirror as shown. The tunable mirror 315 may (i) include two resonant elements, e.g., two ring resonators 335 and may (ii) be tunable over wide range using the vernier effect. Each of the ring resonators may operate as (e.g., be) an optical comb filter; they may be connected in cascade (e.g., by the straight waveguide that is adjacent to the two ring resonators 335). An optical power splitter (e.g., an evanescent coupling or multimode interferometer based 50/50 splitter, illustrated schematically as a “Y” splitter in FIG, 3B) may connect the two ring resonators 335 to the gain section 320 so that a first portion of the light exiting the gain section 320 may propagate (i) through the splitter, (ii) through a first one of the ring resonators 335,(iii) through a second one of the ring resonators 335, and back to the gain section 320, and a second portion of the light exiting the gain section 320 may propagate (i) through the splitter, (ii) through the second one of the ring resonators 335,(iii) through the first one of the ring resonators 335, and back to the gain section 320. For example, the two ring resonators 335 may have different radii, R1 and R2 as shown, and they may be separately tunable by adjusting their respective temperatures using a local heater at each one of the ring resonators 335. The radii R1 and R2 may be selected (e.g., selected to be slightly different) such that if the resonators are tuned so that each is resonant at a desired wavelength of operation, then there is no other wavelength at which the gain section has sufficient gain to result in round-trip gain (within the laser) greater than 1. In some embodiments, different resonant elements, e.g., two optical comb filters, such as vernier sampled gratings, are employed instead of the ring resonators illustrated in FIG. 3B. The gain section 320 provides light amplification to compensate for the round-trip loss, and the phase tuning section includes a phase modulator which may be used to adjust (e.g., under the control of the microcontroller unit 245) the resonant wavelength of the lasing cavity Fabry-Perot (FP) mode to be the same as the wavelength of the overlapping resonant peak of the two ring resonators.

The adjusting of the resonant wavelength of the lasing cavity Fabry-Perot (FP) mode and of the overlapping resonant peak of the two ring resonators may be performed based on measurements, performed at the time of construction, using a wavemeter (as discussed in further detail below) and a power meter. These measurements may involve measuring the laser output power, lasing wavelength, and side mode suppression ratio (SMSR) as a function of heater currents applied to the ring resonators 335 and phase tuning section 325. The resonant wavelength may then be set, during operation, by setting the heater current of the heater for the lasing cavity (e.g., the heater for the phase tuning section 325), and setting the heater current of the heater for the two ring resonators to respective values corresponding to a desired wavelength.

In some embodiments, the comb filters (e.g., one or both of the ring resonators 335, each of which may have an input port and output port) may be positioned elsewhere in the laser of FIG. 3B. For example, one or both of the ring resonators 335 may be incorporated into the output mirror 330. For example, the waveguide of the loop mirror 330 of FIG. 3B may be broken, one end of the break may couple to the input port of a first ring resonator 335, and the other end of the break may couple to the output port of the first ring resonator 335, so that light propagating around the loop may propagate through the first ring resonator. In such an embodiment the second ring resonator 335 of the vernier ring resonators 335 may remain in the back mirror 315. In other embodiments, both resonators 335 of the vernier ring resonators 335 are (connected in cascade) in the loop mirror 330.

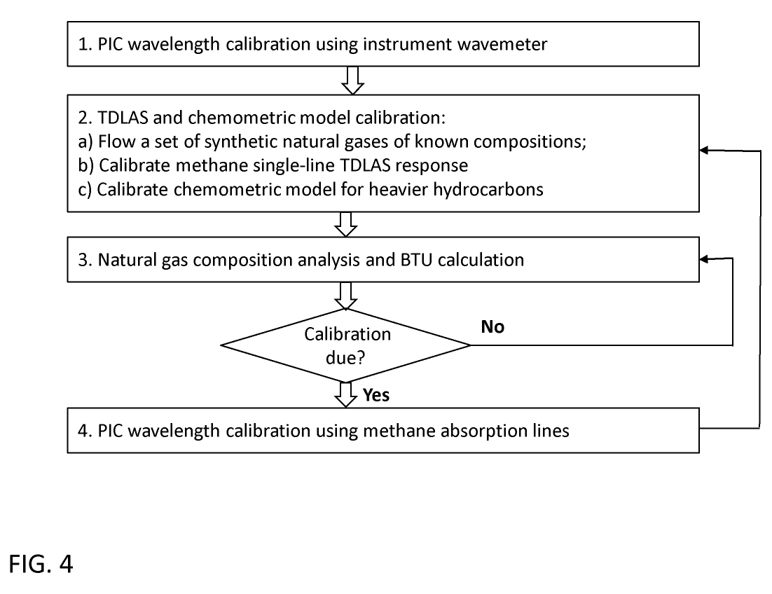

The spectrometric natural gas analyzer may be operated according to the flow chart shown in FIG. 4. The lasing wavelength of the PIC as a function of its tuning parameters can be mapped at the time of construction (e.g., at the factory) using an instrument wavelength meter. Molecules in natural gas can be grouped into two categories. Light molecules with sharp absorption lines such as methane can be characterized with single-line analysis. For example, the transmission fraction at the peak of or integrated over a methane absorption line may be measured, and the molecular density of methane may be determined from this measurement. For example the wavelength may be swept across the absorption peak (e.g., the transmission fraction may be measured at at least 3 (e.g., at least 5) wavelengths within the absorption line). The broad spectra of ethane, propane, and heavier hydrocarbons may also be sampled and analyzed using multivariate analysis such as PLSR. For example, after (or before) measuring the molecular density of methane, a plurality of transmission fraction measurements may be made, each at a wavelength selected so that the effect of methane on the measured transmission fraction is small (e.g., selected not to be within any methane absorption line, or not to be within any strong methane absorption line). The molecular densities of other hydrocarbons may then be estimated from the measured transmission fractions using multivariate analysis, e.g., PLSR . Both for methane and for the other hydrocarbons, the measured transmission fraction or the reference spectral transmission fraction may be adjusted based on the measured pressure or temperature of the gas cell 220. Alternatively, a neural network model may capture the relationship between the spectrometric response and the desired metric, such as gas composition or thermal energy. In some embodiments, relative transmission fractions are measured and used to calculate the relative molecular densities of methane and of other hydrocarbons; in such an embodiment knowledge of the absolute transmission fractions may be unnecessary.

The natural gas analyzer may be calibrated as needed, or on a regular schedule, to correct any potential drift. The absorption features of gas molecules, such as methane, can be used as wavelength reference to compensate for a small drift from the initial calibration. For example, in the embodiment of FIG. 3A, each nominal wavelength may be selected to be near, or centered on, a respective absorption line of methane. During calibration, the laser current at which the transmission fraction is minimum may be found, and used as a reference for determining the current to be used to generate a desired wavelength. For example, the rate of change of wavelength with current, dλ/dI, may be measured at the time of construction of the natural gas analyzer, and the current needed to produce light with a wavelength λ₁ may be calculated as I₀ + (λ₁ – λ₀)/(dλ/dI), where I₀ is the current that generates (as determined from the minimum in the transmission fraction) the wavelength λ₀. The output laser power of each laser (which may change, e.g., as a result of aging) may be calibrated by (i) using valves 216 to fill the gas cell 220 with a calibration gas of a known composition and transmission fraction (e.g., a sample of a standard natural gas mixture, inert gas (e.g., N2 He, H2), or air), from a reservoir 218, or (in the case of air) from the surrounding atmosphere, and (ii) calculating, for each wavelength generated by the light source 205, a calibration factor that when multiplied by a raw (uncalibrated) transmission fraction measured by the natural gas analyzer, results in a calibrated transmission fraction that is equal to the transmission fraction expected for the standard gas sample. If air is used as a calibration gas, it may be drawn from the surrounding atmosphere (e.g., through a filter) by a suitable pump and supplied to the gas cell 220. In such an embodiment, the natural gas analyzer may use no consumables.

Although some embodiments described herein may be used to characterize natural gas, in other embodiments similar or analogous systems and methods may be used to characterize other mixtures of gases.

According to an embodiment of the present disclosure, there is provided a system, including: a natural gas analyzer, including: a coherent light source; a sample chamber configured to contain a sample of natural gas; and a photodetector, the photodetector being configured to measure a transmission fraction, through the sample chamber, of light from the coherent light source; the coherent light source being configured to selectively emit light at a first wavelength, a second wavelength, and a third wavelength; the first wavelength and the second wavelength being within the 1/e² width of a methane absorption line; the third wavelength differing from the first wavelength by at least 5 nm; and the natural gas analyzer being configured to measure the transmission fraction of the sample chamber at the first wavelength, the second wavelength, and the third wavelength. For example, as discussed above, the natural gas analyzer may measure the transmission fraction of the sample of natural gas at two or more (e.g., at 3 or more or at 5 or more) wavelengths within (e.g., at wavelengths within the 1/e² width of) a methane absorption line, and these measurements may be used to calculate the concentration of methane. The natural gas analyzer may further perform measurements at other wavelengths, separated by at least 5 nm from the methane absorption line, which may be used to calculate the respective concentrations of other alkanes or groups of alkanes (e.g., to measure the concentration of each alkane with a carbon number n between 1 and 5, and to measure or estimate the total concentration of all alkanes with a carbon number of 6 or greater).

In some embodiments, the coherent light source includes: a first laser, configured to emit light at the first wavelength and at the second wavelength; a second laser, configured to emit light at the third wavelength; and a wavelength multiplexer for combining light emitted by the first laser with light emitted by the second laser. In some embodiments, the system further includes a photonic integrated circuit, including the first laser, the second laser, and the wavelength multiplexer. In some embodiments, the system further includes a processing circuit. In some embodiments, the processing circuit is configured to turn on or off the first laser and the second laser. For example, the processing circuit may turn on one laser at a time of a plurality of lasers, each being configured to emit at a respective wavelength or within a respective wavelength range.

In some embodiments, the processing circuit is configured to control the wavelength of the first laser, e.g., by adjusting the laser drive current. In some embodiments, the system includes eight lasers including the first laser and the second laser. In some embodiments, the system includes 15 lasers including the first laser and the second laser.

In some embodiments, the first wavelength is between 1620 nm and 1850 nm, and the third wavelength is between 1620 nm and 1850 nm. In some embodiments, the first wavelength is between 1650 nm and 1720 nm, and the third wavelength is between 1650 nm and 1720 nm. In some embodiments, the system is configured to calibrate each of the first wavelength, the second wavelength, and the third wavelength against a methane absorption line. For example, the system may calibrate the first and second wavelengths against a first methane absorption line, and the system may calibrate the third wavelength against a second methane absorption line.

In some embodiments, the system is configured to estimate the concentration of alkanes with molecular formula CₙH₂ₙ₊₂, with n ranging from 1 to 5 including isomers. For example, the system may measure the concentration of each alkane molecule with a carbon number n between 1 and 5 inclusive, e.g., the system may separately measure the respective concentration of each isomer within this set of alkane molecules.

In some embodiments, the coherent light source includes a continuously tunable laser, the continuously tunable laser being capable of emitting light at each of the first wavelength, the second wavelength, and the third wavelength. In some embodiments, the continuously tunable laser includes: a semiconductor gain medium; and a first mirror, the first mirror being a tunable mirror. In some embodiments, the first mirror includes a first resonant element and a second resonant element in a vernier configuration. In some embodiments, the system includes a photonic integrated circuit, including: the semiconductor gain medium; the first mirror; a phase tuning section; and a second mirror.

In some embodiments, the system further includes a temperature control circuit for controlling the temperature of the photonic integrated circuit. In some embodiments, the system further includes a processing circuit. In some embodiments, the processing circuit is configured to control the wavelength of the continuously tunable laser.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y%” of a first number, it means that the second number is at least (1-Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed herein could be termed a second element, component, region, layer or section, without departing from the spirit and scope of the inventive concept.

Spatially relative terms, such as “beneath”, “below”, “lower”, “under”, “above”, “upper” and the like, may be used herein for ease of description to describe one element or feature’s relationship to another element(s) or feature(s) as illustrated in the figures. It will be understood that such spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

As used herein, the term “major component” refers to a component that is present in a composition, polymer, or product in an amount greater than an amount of any other single component in the composition or product. In contrast, the term “primary component” refers to a component that makes up at least 50% by weight or more of the composition, polymer, or product. As used herein, the term “major portion”, when applied to a plurality of items, means at least half of the items. As used herein, any structure or layer that is described as being “made of” or “composed of” a substance should be understood (i) in some embodiments, to contain that substance as the primary component or (ii) in some embodiments, to contain that substance as the major component.

It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the inventive concept refers to “one or more embodiments of the present disclosure”. Also, the term “exemplary” is intended to refer to an example or illustration. As used herein, the terms “use,” “using,” and “used” may be considered synonymous with the terms “utilize,” “utilizing,” and “utilized,” respectively.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of "1.0 to 10.0" or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1 – 35/100) times 10) and the recited maximum value of 13.5 (i.e., (1 + 35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

It will be understood that when an element is referred to as being “directly connected” or “directly coupled” to another element, there are no intervening elements present. As used herein, “generally connected” means connected by an electrical path that may contain arbitrary intervening elements, including intervening elements the presence of which qualitatively changes the behavior of the circuit. As used herein, “connected” means (i) “directly connected” or (ii) connected with intervening elements, the intervening elements being ones (e.g., low-value resistors or inductors, or short sections of transmission line) that do not qualitatively affect the behavior of the circuit.

Although exemplary embodiments of a system and method for the analysis of natural gas have been specifically described and illustrated herein, many modifications and variations will be apparent to those skilled in the art. Accordingly, it is to be understood that a system and method for the analysis of natural gas constructed according to principles of this disclosure may be embodied other than as specifically described herein. The invention is also defined in the following claims, and equivalents thereof.

What is claimed is:

1. A system, comprising:
a natural gas analyzer, comprising:
a coherent light source;
a sample chamber configured to contain a sample of natural gas; and
a photodetector,
the photodetector being configured to measure a transmission fraction, through the sample chamber, of light from the coherent light source;
the coherent light source being configured to selectively emit light at a first wavelength, a second wavelength, and a third wavelength;
the first wavelength and the second wavelength being within the 1/e² width of a methane absorption line;
the third wavelength differing from the first wavelength by at least 5 nm; and
the natural gas analyzer being configured to measure the transmission fraction of the sample chamber at the first wavelength, the second wavelength, and the third wavelength.

2. The system of claim 1, wherein the coherent light source comprises:
a first laser, configured to emit light at the first wavelength and at the second wavelength;
a second laser, configured to emit light at the third wavelength; and
a wavelength multiplexer for combining light emitted by the first laser with light emitted by the second laser.

3. The system of claim 2, comprising a photonic integrated circuit, comprising the first laser, the second laser, and the wavelength multiplexer.

4. The system of claim 2, further comprising a processing circuit.

5. The system of claim 4, wherein the processing circuit is configured to turn on or off the first laser and the second laser.

6. The system of claim 4, wherein the processing circuit is configured to control the wavelength of the first laser.

7. The system of claim 2, comprising eight lasers including the first laser and the second laser.

8. The system of claim 2, comprising 15 lasers including the first laser and the second laser.

9. The system of claim 1, wherein the first wavelength is between 1620 nm and 1850 nm, and the third wavelength is between 1620 nm and 1850 nm.

10. The system of claim 9, wherein the first wavelength is between 1650 nm and 1720 nm, and the third wavelength is between 1650 nm and 1720 nm.

11. The system of claim 1, wherein the system is configured to calibrate each of the first wavelength, the second wavelength, and the third wavelength against a methane absorption line.

12. The system of claim 1, wherein the system is configured to estimate the concentration of alkanes with molecular formula CₙH₂ₙ₊₂ with n ranging from 1 to 5 including isomers.

13. The system of claim 1, wherein the coherent light source comprises a continuously tunable laser, the continuously tunable laser being capable of emitting light at each of the first wavelength, the second wavelength, and the third wavelength.

14. The system of claim 13, wherein the continuously tunable laser comprises:
a semiconductor gain medium; and
a first mirror,
the first mirror being a tunable mirror.

15. The system of claim 14, wherein the first mirror comprises a first resonant element and a second resonant element in a vernier configuration.

16. The system of claim 14, comprising a photonic integrated circuit, comprising:
the semiconductor gain medium;
the first mirror;
a phase tuning section; and
a second mirror.

17. The system of claim 16, further comprising a temperature control circuit for controlling the temperature of the photonic integrated circuit.

18. The system of claim 13, further comprising a processing circuit.

19. The system of claim 18, wherein the processing circuit is configured to control the wavelength of the continuously tunable laser.
